(19) United States
(12) Patent Application Publication
(10) Pub. No.: US 2019/0125979 A1
(43) Pub. Date: May 2, 2019

(54) SAFETY HYPODERMIC SYRINGE AND NEEDLE

(71) Applicant: Anthony DiPasquale, Burlington, NC (US)

(72) Inventor: Anthony DiPasquale, Burlington, NC (US)

(21) Appl. No.: 15/801,629

(22) Filed: Nov. 2, 2017

(51) Int. Cl. A61M 5/32; A61M 5/315

(52) U.S. Cl. CPC A61M 5/3221; A61M 5/34; A61M 2005/3247; A61M 5/31501

(57) ABSTRACT

A safety syringe has at least two angled lock extensions on opposing sides of a plunger shaft. A plunger tip integral with the shaft has at least a pair of locking lugs. A syringe barrel has a needle assembly housing end securing the needle assembly in place for injections. The needle assembly has a needle secured in a needle housing and at least two locking ledges formed as an integral part. The end cap further comprises an opening sized to receive the shaft to fit and move back and forth, and at least two locking edges. The locking edges engage with the angled lock extensions to lock the plunger in place when fully withdrawn. The locking ledges on the needle housing engage with the locking lugs when the plunger tip is pushed into the needle housing, so as to use to retract the needle into the barrel.

### CROSS-REFERENCE TO RELATED APPLICATION(S)

Not applicable

### BACKGROUND OF THE INVENTION

### 1. Field of Invention

The present invention relates to the field of syringes, more particularly to safety hypodermic syringe needles.

### 2. Description of Related Art

In recent years, health care professionals and treatment facilities have increasingly been concerned with used needle sticks. Needle sticks pose a constant danger in all treatment settings. Even the most careful care giver can end up getting stuck by a used needle. Patients can sneeze, go into a seizure, be combative, or the health care specialist can be accidentally jostled, or they can stumble. Circumstance occur on a frequent basis where a needle accidentally makes contact.

Further, some medical facilities can be managed by incompetent managers behaving irresponsibly or criminally, attempting to repackage and use as new used needles or simply mishandle disposal.

With the increased attention, there has been an increasing demand for safe syringes that minimize the chances of a needle stick. Numerous solutions have been tried and are available. Improvements are constantly desire, especially solutions that minimize the time that a used needle remains exposed and minimize physical manipulation.

Based on the foregoing, there is a need in the art a system, which will facilitate a quick and simple method to remove a needle's exposure after use.

### SUMMARY OF THE INVENTION

A safety syringe comprised of a plunger having a shaft with a plunger seal at one end and a plunger end cap proximate to an opposite end, and having at least two angled lock extensions on opposing sides of said shaft. The plunger tip is integral with the shaft having with at least a pair of locking lugs. A syringe barrel with a needle assembly housing end and comprising an end cap at an opposite end for receiving the shaft, and wherein the assembly housing end secures the needle assembly in place for injections. A needle assembly having a needle secured in a plastic needle housing and at least two locking ledges formed as an integral part of said needle housing. The end cap further comprises an opening sized to receive the shaft to fit and move back and forth, and at least two locking edges. The locking edges engage with the angled lock extensions to lock the plunger in place when fully withdrawn. The locking ledges on the needle housing engage with the locking lugs when the plunger tip is pushed into the needle housing, so as to use to withdraw the needle assembly and the needle, and thus retract the needle into the barrel.

The locking lugs comprise two hemispheric halves separated by an open slit allowing the locking lugs to move closer together under force to enter into the needle housing and engage with the locking ledges to securely mate with the needle housing.

The needle housing comprises a surrounding rubber-like O-ring proximate to an edge.

The needle is molded into the plastic needle housing.

The safety syringe further comprises injecting medication, pushing forward on the plunger to lock the plunger and needle assembly together, and then immediately withdrawing the plunger and thus pull the needle assembly back and into the barrel, never exposing the used needle.

The safety syringe further comprising pulling the plunger back till the angled lock extensions engage with the locking edges.

A method of securing a used needle in a syringe, comprising the steps of operating a syringe by pushing forward a plunger to move a plunger shaft down a barrel to compress down to inject medication through to compress down to inject medication through a needle, pushing the plunger shaft to push a plunger seal down and into a needle housing, where the outer sides of a plunger tip compresses to enter the interior of a needle housing and engage a locking ledge with locking lugs, creating a locked connection that essentially permanently mates the needle assembly with the plunger shaft. After mating the plunger shaft and needle assembly, the plunger is withdrawn back from a needle assembly housing end of the barrel to pull needle housing back and withdrawing needle into an interior of the barrel. Withdrawing the plunger fully back beyond a plunger cap, with a sloping structure of an angled lock extensions on the plunger shaft depressed while sliding past end cap to spring outward and engage locking edges on the plunger end cap to lock the needle secure inside the barrel

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a side and cross sectional view of the syringe plunger, according to an embodiment of the present invention;

FIG. 2 is a side cutaway view of the needle housing of the syringe, according to an embodiment of the present invention;

FIG. 3 is a side cutaway view of the barrel of the syringe, according to an embodiment of the present invention.

### DETAILED DESCRIPTION OF PREFERRED EMBODIMENTS

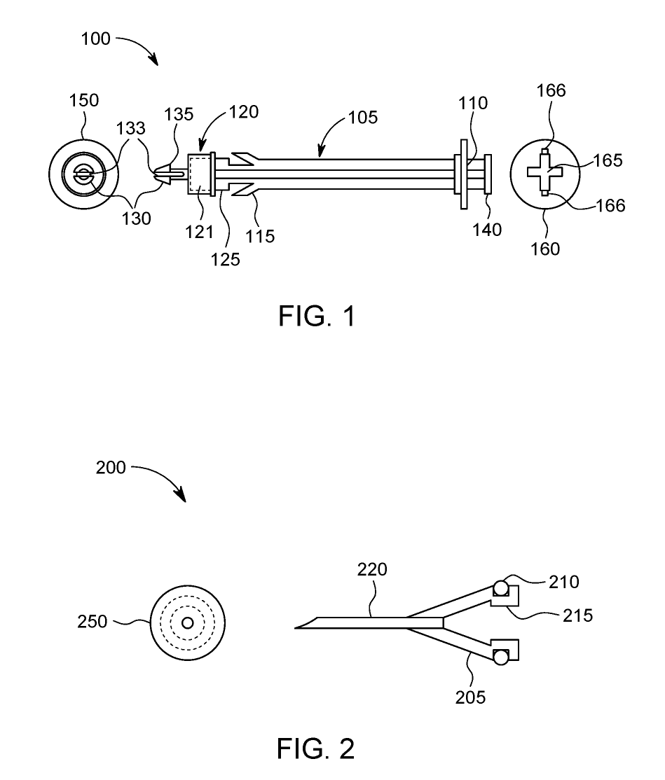

Preferred embodiments of the present invention and their advantages may be understood by referring to FIGS. 1-3, wherein like reference numerals refer to like elements.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

The present invention will now be described in detail with reference to embodiments thereof as illustrated in the accompanying drawings.

Referring to FIG. 1, as depicted, the syringe according to an embodiment can include a plunger 100 having a plunger shaft 105 with an endcap 110. The shaft 105 can include at least a pair of angled lock extensions 115. A plunger seal 120, made from a pliant plastic, polyurethane, or the like, acts to provide a chamber formed by the seal 120 in fluid-proof contact with a syringe barrel 305 to contain an injectable medication. The plunger seal 120 as depicted can include a receptacle 121 able to receive an attachment 125 that can collapse into the receptacle 121 when pushed forward inside barrel 305.

The plunger tip 130 can include two semicircular locking lugs 135 comprised of locking lips that fit into a matching set of ledges 215 in a needle housing 205. The semicircular locking lugs 135 essentially form two cross-sectional hemispheric halves 150 separated by an open slit 133 designed to allow the two locking lugs 135/cross-sectional hemispheric halves 150 to move closer together under force to enter into and securely mate with needle housing 205. The plunger end 140 opposite from plunger tip 130 provides a finger rest to press against the plunger and inject medication.

The end view 160 of plunger end cap shows a “+” shaped opening 165 within which plunger shaft 105 can fit and move back and forth. The plunger end cap 110 includes locking edges 166 into which angled lock extensions 115 can fit and lock plunger 100 in place relative to syringe barrel 300.

FIG. 2 shows a needle assembly 200 for the syringe. Needle assembly 200 can include a needle housing 205. The needle housing 205 can be comprised of a solid, plastic molded body that includes a surrounding rubber-like O-ring 210 proximate to the edge providing a tight fit fluid-proof when inserted into the barrel 305 of the syringe. The needle housing 205 can also include a locking ledge 215 formed as an integral part of the needle housing 205. Needle 220 can be molded directly inside needle housing 205. A cross section view 250 shows a view from the front of the needle housing 205.

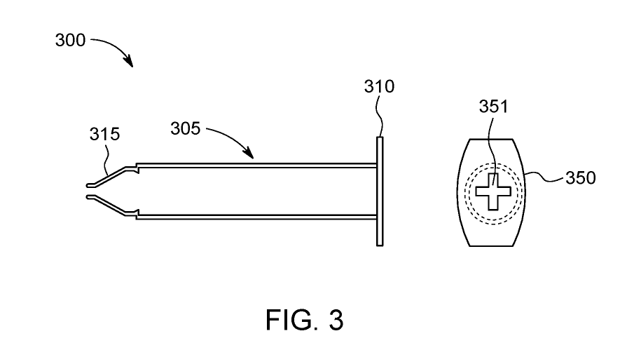

In FIG. 3, a syringe barrel 300 is shown and can include barrel 305, typically made from a clear or translucent hard plastic. Barrel 305 is hollow and includes an end cap 310 and a needle assembly housing end 315. End cap view 350 shows how the end cap 310 include a “+” opening 351 for receiving plunger shaft 105, which also secures plunger end cap 110 in place. The needle assembly housing end 315 receives the needle assembly 200 and secures the needle assembly 200 in place for rejections.

In an embodiment, the disclosed syringe operates by pushing forward the plunger 100 inward moving the plunger shaft 105 to compress down to inject medication through needle 220. As the plunger shaft 105 pushes the plunger seal 120 down and into the needle housing 205, the outer sides of plunger tip 130 compresses the hemispherical halves 150 to enter the interior of needle housing 205 and engage locking ledge 215 with locking lugs 135. This locked connection essentially permanently mates needle assembly 200 with plunger 100.

After mating plunger 100 and needle assembly 200, the plunger is withdrawn back from needle assembly housing end 315, which in turns pulls needle housing 205 back withdrawing needle 220 into the interior of barrel 305 where in cannot make contact and cause a needle stick. As the plunger 100 is fully withdrawn back beyond plunger cap 110, the sloping structure of angled lock extensions 115, depressing the angled lock extensions 115 till sliding past end cap 110 to spring outward and engage locking edges 166 of plunger end cap 110. This mechanical locking action locks the needle secure inside the barrel 305, making impossible to accident extend the needle outward and pose a danger.

In an embodiment, to use the disclosed syringe, all a health care professional needs to do is inject medication, pushing forward to lock the plunger 100 and needle assembly 200 together, and then immediately withdraw the plunger 100 and thus pull the needle assembly 200 back from the patient and into the barrel 300, never exposing the used needle.

The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

I claim:

1. A safety syringe comprising:
a plunger having a shaft with a plunger seal at one end and a plunger end cap proximate to an opposite end, and having at least two angled lock extensions on opposing sides of said shaft;
a plunger tip integral with the shaft having at least a pair of locking lugs;
a syringe barrel with a needle assembly housing end and comprising an end cap at an opposite end for receiving the shaft, and wherein the assembly housing end secures the needle assembly in place for injections;
a needle assembly having a needle secured in a plastic needle housing and at least two locking ledges formed as an integral part of said needle housing;
wherein the end cap further comprises an opening sized to receive the shaft to fit and move back and forth, and at least two locking edges;
wherein the locking edges engage with the angled lock extensions to lock the plunger in place when fully withdrawn; and
wherein the locking ledges on the needle housing engage with the locking lugs when the plunger tip is pushed into the needle housing, so as to use to withdraw the needle assembly and the needle, and thus retract the needle into the barrel.

2. The safety syringe of claim 1, wherein the locking lugs comprises two hemispheric halves separated by an open slit allowing the locking lugs to move closer together under force to enter into needle housing and engage with the locking ledges to securely mate with needle housing.

3. The safety syringe of claim 1, wherein the needle housing comprises a surrounding rubber-like O-ring proximate to an edge.

4. The safety syringe of claim 1, wherein the needle is molded into the plastic needle housing.

5. The safety syringe of claim 1, further comprising steps of:
injecting medication;
pushing forward on the plunger to lock the plunger and needle assembly together;
then immediately withdrawing the plunger and thus pull the needle assembly back and into the barrel, never exposing the used needle.

6. The safety syringe of claim 5, further comprising step of:
pulling the plunger back till the angled lock extensions engage with the locking edges.

7. A method of securing a used needle in a syringe, comprising the steps of:
operating a syringe by pushing forward a plunger to move a plunger shaft down a barrel to compress down to inject medication through a needle;
pushing the plunger shaft to push a plunger seal down and into a needle housing, where the outer sides of a plunger tip compresses to enter the interior of a needle housing and engage a locking ledge with locking lugs, creating a locked connection that essentially permanently mates the needle assembly with the plunger shaft;
after mating the plunger shaft and needle assembly, the plunger is withdrawn back from a needle assembly housing end of the barrel to pull the needle housing back and withdrawing the needle into an interior of the barrel;
withdrawing the plunger fully back beyond a plunger cap, with a lock extensions on the plunger shaft depressed while sliding past end cap to engage locking edges on the plunger end cap to lock the needle secure inside the barrel.
